(19) United States
(12) Patent Application Publication
(10) Pub. No.: US 2019/0133827 A1
(43) Pub. Date: May 9, 2019

(54) EYE MASK FOR PROTECTION OF EYELASHES

(71) Applicant: Mind & Body Beauty, LLC, Winnetka, CA (US)

(72) Inventor: Jason W. Carver, Winnetka, CA (US)

(21) Appl. No.: 15/804,966

(22) Filed: Nov. 6, 2017

(51) Int. Cl. A61F 9/04

(52) U.S. Cl. CPC A61F 9/04

(57) ABSTRACT

An eye mask assembly includes a mask, a strap, and a pair of lash-protection rings. The mask has a first end and a second end connected by a bridge, and a first surface opposite a second surface. The strap connects with the first and second ends of the mask. The lash-protection rings are connected to the first surface of the mask and are configured to elevate the first surface of the mask away from a user's eyelashes when the eye mask assembly is worn by a user.

### TECHNICAL FIELD

Examples described herein relate to an eye mask that protect a user's eyelashes.

### BACKGROUND

A current beauty trend is for a user to wear temporary or permanent eyelash extensions. In some instances, an individual eyelash extension (an artificial lash) may be connected to a user's individual, real eyelash. In other instances, a user may wear an assembly of artificial eyelashes that are connected to multiple individual eyelashes of the user or to the skin adjacent the eyelashes.

In some instances, the artificial lash will remain connected to the individual eyelash until the individual eyelash naturally falls out at the end of its growing cycle. In some instances, the artificial lash will remain connected to the individual lash for weeks, if not at least a month. However, this connection is somewhat fragile, as the individual eyelash is a short, slender, and lightweight object. The addition of the artificial lash may put additional stress on the connection of the eyelash to the eyelid. This may cause the individual lash to fall out prematurely.

In some instances, the application of artificial lashes is expensive, as the artificial lashes may be specifically engineered, and a skilled person must be engaged to apply the artificial lashes to each individual lash. The application process may be time intensive, and therefore increase the overall application cost and the user's overall investment in the artificial lashes.

In order to maximize the duration (and therefore investment) in which the artificial lash remains connected to the individual lash (which remains connected to the eyelid), the user is typically advised to avoid unnecessary contact or interaction with the artificial eyelashes. Unnecessary contact may weaken the bond between the artificial lash and the user's eyelash, and cause the artificial lash to separate from the individual lash. Unnecessary contact may also weaken the bond between the individual eyelash and the user's eyelid, causing the individual eyelash (with or without the artificial lash connected to it) to fall out prematurely.

To prolong the duration and quality of the artificial eyelash connection, the user may wear an eye mask during sleep, massages, or other period when the eyes may be closed and the eyelashes may come into contact with an object. In some instances, the eye mask may be worn over a user's eyes to protect a user's eyelashes and also to block out light, to protect the delicate skin area that surrounds the eyes, to protect the user's eyes from irritation, such as preventing a ceiling fan's moving air from irritating the eyes, and/or protecting a user's applied makeup from being smudged or coming off on bed sheets, pillows, furniture, or other items.

A user may sleep with an eye mask on. While sleeping or preparing to go to sleep, a user may move their head in various positions. In some instances, a user may sleep on their stomach, be positioned on their side, or sleep on their back with their head turned to the side. These positions may cause the mask to come into contact with an object, such as a pillow case or mattress. This contact may be undesirable, as the contact of the mask with the object may cause the mask to contact or create friction with the user's eyelashes, or place additional forces or friction if the mask already contacts the eyelashes. For example, the user's head may be positioned so that the pillow pushes against the eye socket area of the mask, which then puts pressure on the area surrounding a user's eye socket. This may include putting pressure on or creating friction with the eyelashes. This pressure may damage the eyelashes or cause the premature disconnection of the artificial lash with the individual lash, or the individual lash from the eyelid.

In other examples, the length of the user's eyelashes (either with or without the artificial lash connected) may contact an inner surface of the eye mask. This contact may cause irritation to the user, damage the eyelashes, and/or cause the premature disconnection of the artificial lash with the individual lash, or the individual lash from the eyelid.

The information included in this Background section of the specification, including any references cited herein and any description or discussion thereof, is included for technical reference purposes only and is not to be regarded subject matter by which the scope of the invention as defined in the claims is to be bound.

### SUMMARY

In one implementation, an eye mask assembly includes a mask, a strap, and a pair of ring-shaped bolsters. The mask may have a first end and a second end connected by a bridge, and a first surface opposite a second surface. The strap may connect the first and the second ends of the mask. The ring-shaped bolsters may be connected to the first surface of the mask and configured to elevate the first surface of the mask away from a user's eyelashes when the eye mask assembly is worn by a user.

In another implementation, an eye mask assembly may include a mask, a strap, and lash protection rings. The mask has a first end and a second end connected by a bridge, and a first surface opposite a second surface. The strap connects with the first and second ends of the mask. The lash-protection rings are connected to the mask and extend away from the first surface of the mask. Each lash-protection ring has an inner wall and a top end, wherein the top end of the lash-protection ring forms a skin-contact region of the eye mask assembly. A cavity is formed by the inner wall of the lash-protection ring and the first surface of the mask. The eye mask assembly is configured to be worn by a user such that the cavity is configured to be positioned above and around a user's eye socket to prevent a user's eyelashes from contacting the inner wall or the first surface of the mask. The skin-contact region is configured to contact an area of skin surrounding the user's eye sockets. The first surface of the mask is spaced apart from the top end to avoid contact with the user.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments and implementations and illustrated in the accompanying drawings.

### BRIEF DESCRIPTION OF THE DRAWINGS

Features, aspects and attendant advantages of described embodiments will become apparent from the following detailed description, in which:

FIG. 1 is a top view of an embodiment of an eye mask assembly.

FIG. 2 is a partial top isometric view of the eye mask assembly of FIG. 1.

FIG. 3 is a partial cross-sectional view along line 3-3 of the eye mask assembly of FIG. 2.

FIG. 4 is a partial side isometric view of the eye mask assembly of FIG. 1.

### DETAILED DESCRIPTION

Examples described herein include devices, methods, and systems for a protective eye mask that does not undesirably contact a user's eyelashes when it is worn. To overcome the potential undesired contact with the eyelashes, an eye mask with lash-protection rings may be desired. In some examples, the eye mask includes two lash-protection rings. Each lash-protection ring elevates the eye mask away from a user's eye socket and eyelashes. Each lash-protection ring may be formed from a flexible material so that it may conform to the shapes of various users' faces. In some examples, the lash-protection ring has a core that is covered with a layer of material. The layer of material may be formed with a material suitable to be positioned adjacent the delicate skin adjacent to the user's eyes. For the purposes of this disclosure, the word “ring” when used in the term “lash-protection ring” is not meant to limit the structure to a purely circular shape, but is rather encompasses non-circular, curved loops as well, including substantially oval and obround shapes.

When worn by a user, in some examples, the skin-contact region of each lash-protection ring contacts the area surrounding the user's eye sockets, thereby creating a large cavity of space between an inside surface of the lash-protection ring, an inside surface of the eye mask, and the user's eyelids. This large cavity protects the user's eyelashes, as it prevents the user's eyelashes from inadvertent contact with or irritation from pillows, mattress, furniture, or sheets, and also from contacting the inner surface of the mask when the mask contacts the pillows, mattress, furniture, or sheets.

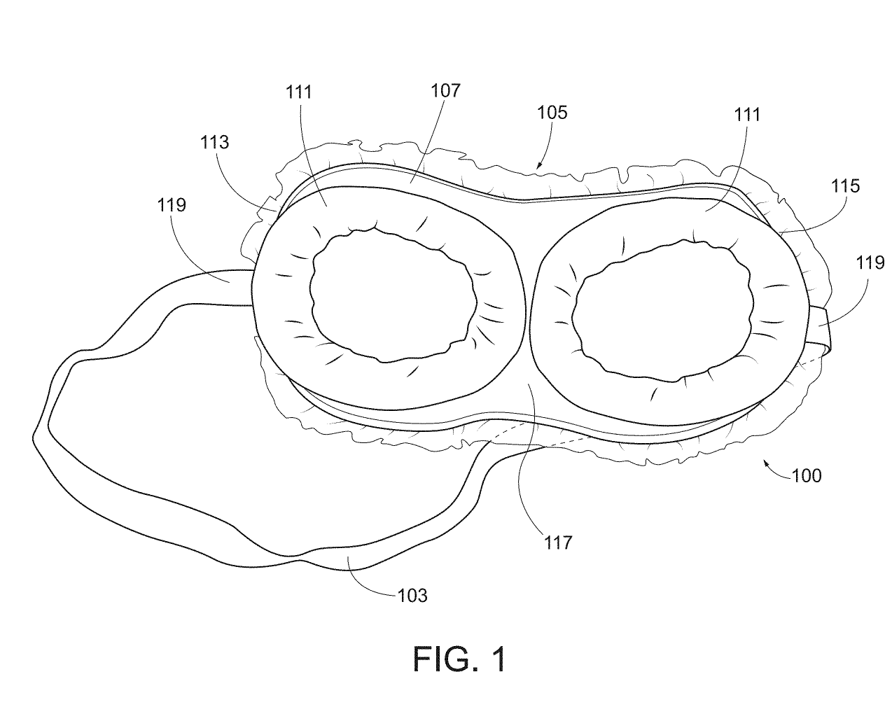

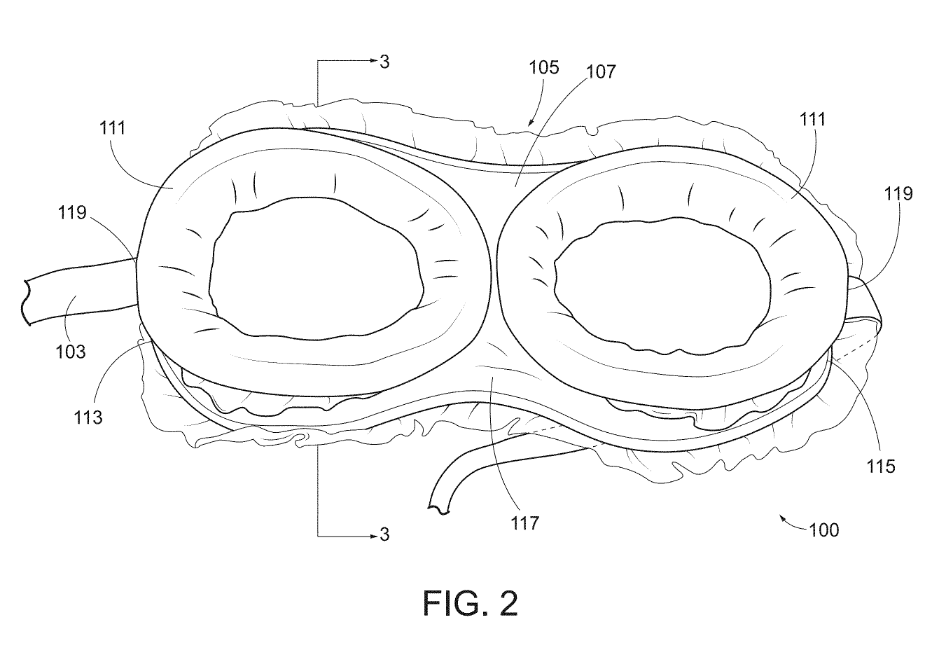

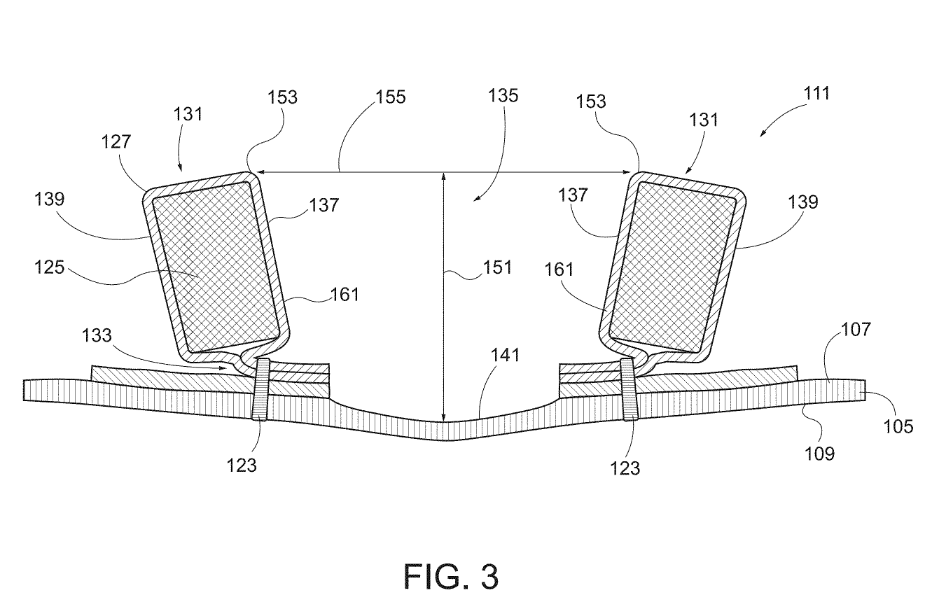

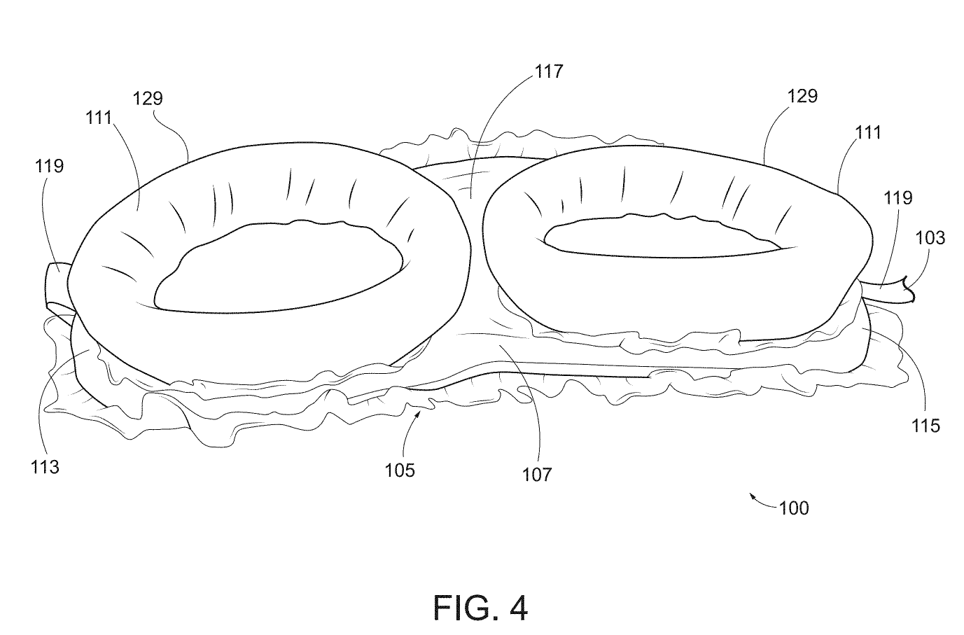

FIG. 1 is a top view of an example eye mask assembly 100. FIG. 2 is a partial top isometric view of the eye mask assembly 100. FIG. 3 is a partial cross-sectional view along line 3-3 of the eye mask assembly of FIG. 2. FIG. 4 is a partial side isometric view of the eye mask assembly 100. The eye mask assembly 100 may include a mask 105, two lash-protection rings 111, and a strap 103.

As shown in FIGS. 1, 2, and 4, the mask 105 has a first surface 107 with a second surface 109 (see FIG. 3) opposite the first surface 107. The mask 105 may be defined by a first end 113 opposite a second end 115, and a bridge 117 positioned between the first end 113 and the second end 115. In some examples, the mask 105 may have a length that is generally longer than the width. The first end 113 and second end 115 may be curved. In some examples, the bridge 117 may have a width that is narrower than a maximum width of the first end 113 and the second end 115. In some examples, an overall length of the mask 105 may be slightly curved. In some examples, the mask 105 may be formed from a single layer of material. In other examples, the mask 105 may be formed from multiple layers of material.

As shown in FIGS. 1, 2, and 4, in some examples, the eye mask assembly 100 includes the strap 103. The strap 103 may be an elastic band that may be partially formed from fabric. In other embodiments, the strap 103 may be adjustable in length through the use of clips, buckles, graduated snap interfaces, and other commonly known structures. Ends 119 of the strap 103 are connected to the first end 113 and the second end 115 of the mask 105. In examples where the mask 105 is formed from multiple layers of material, the ends 119 may be positioned in between the first surface 107 and the second surface 109, such that the ends 119 are between the layers of material that form the mask 105. Positioning the ends 119 between the first surface 107 and second surface 109 may help prevent the ends 119 from contacting the user's face when the mask is worn. This may help prevent skin or eye irritation or undesired skin creases. The ends 119 may be connected and secured to the first end 113 and the second end 115 of the mask 105 through a sewn stitch, adhesive, snap, or other connection method.

As shown in FIGS. 1-4, the eye mask assembly 100 includes lash-protection rings 111 positioned adjacent to the mask 105. The lash-protection rings 111 may be understood as ring-shaped bolsters connected to the first surface 107 of the mask 105 and configured to elevate the first surface 107 of the mask 105 away from a user's eyelashes when the eye mask assembly 100 is worn by a user. As shown in FIG. 3, each lash-protection ring 111 may have a top end 131 positioned opposite a bottom end 133. In some examples, the bottom end 133 of the lash-protection ring 111 is positioned adjacent to the first surface 107 of the mask 105. In some examples, each lash-protection ring 111 may have a generally oval or obround shape. The oval or obround shape of the lash-protection ring 111 may complement the general shape of a user's eye socket. As shown in FIG. 3, the lash-protection ring 111 may have an outer wall 139 and an inner wall 137 that form an outer perimeter and an inner perimeter of the lash-protection ring 111.

As shown in FIG. 3, in some examples, each lash-protection ring 111 may be formed by a core 125 and a cover 127. In some examples, the core 125 may have a generally rectangular cross-sectional shape. In some examples, the rectangular cross-sectional shape has rounded corners. In other examples, the core 125 may have a cross-sectional shape that is circular, oval, obround, square, triangular, oblong, or other shape.

The core may 125 may be formed from a reversibly deformable, compressible or compactable material, such as foam, sponge, or insulated fabric. The material of the core 125 may be configured to reversibly compress or conform to a variety of eye socket and/or facial shapes of different users while still being stiff or semi-rigid enough to form a protective region around user's eye socket. This incompressible or semi-rigid protective region may be configured to protect the user's eyelashes.

A height and a width of the core 125 help define an overall height and width of the lash-protection ring 111. In some examples, the core 125 is formed into an oval or obround shape that generally controls the shape of the lash-protection ring 111. As shown in FIGS. 1-4, in some examples, the core 125 forms a wall-like structure of the obround shape of the lash-protection ring 111 that extends away from the first surface 107 of the mask 105. The core 125 may be sized and positioned so that the inner wall 137 of the lash-protection ring 111 forms a protective perimeter around a user's eye socket and the protective perimeter formed by the inner wall 137 is larger than a periphery of a user's eye socket. The protective perimeter formed by the inner wall 137 may be sized so to prevent the user's eyelashes from contacting the inner wall 137.

In some examples, a securing feature 123 may connect the mask 105 with the lash-protection ring 111. The securing feature 123 may be a sewn stitch, adhesive, hook and loop fastener, snaps, or other similar-type securing feature. In some examples, the cover 127 is connected to the first surface 107 using the securing feature 123. As shown in FIG. 3, the cover 127 may be a flap of material positioned about and generally surrounding the core 125. In some examples, the cover 127 surrounds the core 125 so that the core 125 is not directly connected to the first surface 107 of the mask. In these examples, the securing feature 123 may create a seam 145 between the cover 127 and the mask 105 so that the core 125 is positioned somewhat normal to the first surface 107. The core 125 may then flex along the seam 145 with respect to the first surface 107. This may allow the lash-protection ring 111 to be manipulated to flexibly conform to a user's face shape.

When the components of the eye mask assembly 100 are assembled, a first lash-protection ring 111 may be positioned between the first end 113 and the bridge 117 of the mask 105, and a second lash-protection ring 111 may be positioned between the second end 115 and the bridge 117 of the mask 105. Each lash-protection ring 111 is positioned adjacent to the first surface 107 of the mask 105. In some examples, each lash-protection ring 111 is connected to the first surface 107 of the mask 105. In some examples, as shown in FIG. 3, a layer of material 121 may be positioned between the lash-protection ring 111 and the first surface 107 of the mask.

In some examples, the lash-protection ring 111 and the first surface 107 of the mask 105 form a cavity 135, with the inner wall 137 of the lash-protection ring 111 forming the walls 161 of the cavity 135 and the first surface 107 forming a base 141 of the cavity 135. The top end 131 of the lash-protection ring 111 may form an opening 153 to the cavity 135. The cavity 135 may be configured to protect the user's eye socket and eyelashes from contact with the eye mask assembly 100 and with other objects such as pillows, mattresses, sheets, plane seats, furniture, massage tables, and the like. The cavity 135 may be tapered such that the opening 153 to the cavity 135 is larger than the base 141 of the cavity 135. An outer wall of the lash-protection ring 111 may extend away from the first surface 107 of the mask 105 at an acute angle with respect to the first surface 107 to help form the tapered shape of the cavity 135. The tapered cavity shape may also reduce an overall form factor of the mask 105, making it more comfortable to wear by a user, so that it doesn't unnecessarily extend away from the user's face and undesirably contact a pillow, bed sheet, or other item when the mask assembly 100 is worn by the user.

A depth 151 of the cavity 135 may be sized so that a user's eyelashes will not contact the base 141 of the cavity 135 when the mask assembly 100 is worn by a user. The depth 151 may be generally controlled by a height and a width of the core 125 and an alignment of the core 125 with the first surface 107. The height and width of the core 125 are sized to not be so large as to disrupt the positon of the mask 105 when the mask is positioned on the user's face and the user lays their head on a pillow. An overall width 155 of the cavity 135 at the opening 153 may be sized so that a user's eyelashes will not contact any portion of the wall 161 of the cavity 135 formed by the inner wall 137 of the lash-protection ring 111.

As shown in FIG. 4, when the lash-protection rings 111 are assembled to the mask 105, the top end 133 of each lash-protection ring 111 may form a skin-contact region 129 of the mask assembly 100.

To use the assembly 100, the assembly 100 is positioned on a user's head with the lash-protection rings 111 positioned over a user's eye sockets and the strap 103 wrapped about the backside of a user's head. The lash-protection rings 111 may be configured to elevate the first surface 107 of the mask 105 away from the user's face, eye sockets, and eyelashes. Each cavity 135 of the eye mask assembly 100 may be positioned above and around a user's eye socket and configured to prevent a user's eyelashes from contacting the walls 161 or the base of the cavity 135. When the assembly 100 is positioned on a user's head, the skin-contact region 129 may be positioned adjacent a user's skin and surround the user's eye socket. In some examples, the entire skin-contact region 129 is offset or elevated away from the first surface 107 of the mask 105. In some examples, only the skin-contact region 129 contacts the user's face, and the mask 105 and strap 103 do not contact the user's face. In some examples, the strap 103 may contact a separate portion of the user's head so that the mask may be temporarily secured about a user's head.

In some examples, the lash-protection rings 111 may flex with respect to the mask 105 at a flexion point formed by the securing feature 123 that secures the cover 127 of the lash-protection ring 111 to the first surface 107 of the mask 105. The pivotability of the lash-protection rings 111 with respect to the mask 105 may allow the lash-protection rings 111 to temporarily adjust to fit the various shapes of a user's eye socket and facial shape.

In some examples, the mask assembly 100 is configured to protect the area surrounding a user's eye socket. In some examples, this protection includes protection a user's eyelashes from coming into contact with the eye mask assembly 100, such as contacting the walls 161 of the cavity 153 formed by the inner walls 137 of the lash-protection rings 111, the base 141 of the cavity 153 formed by the first surface 107 of the mask 105, or from contacting other items such as a pillow, bed sheet, massage table, headrest, etc. In some examples, the user's eyelashes should not contact the mask assembly 100 when the user's eyes are closed. In some examples, the user's eyelashes should not contact the mask assembly 100 when the user's eyes are open or closed.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the structures disclosed herein, and do not create limitations, particularly as to the position, orientation, or use of such structures. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. The above detailed description of examples is not intended to be exhaustive or to limit the method and system for protective eyewear to the precise form disclosed above. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. It will be further appreciated that one or more components of protective eyewear may be used in combination with any of the components or assemblies of any of the examples described herein. Changes in detail or structure may be made without departing from the basic elements of the invention as defined in the following claims.

What is claimed is:

1. An eye mask assembly comprising
a mask having a first end and a second end connected by a bridge, and a first surface opposite a second surface;
a strap connecting the first and the second ends of the mask;
a pair of ring-shaped bolsters connected to the first surface of the mask and are configured to elevate the first surface of the mask away from a user's eyelashes when the eye mask assembly is worn by a user.

2. The eye mask assembly of claim 1, wherein the ring-shaped bolsters are substantially oval or obround in shape.

3. The eye mask assembly of claim 1, wherein an outer wall of each of the ring-shaped bolsters extends away from the first surface of the mask at an acute angle with respect to the first surface.

4. The eye mask assembly of claim 1, wherein each of the ring-shaped bolsters defines a cavity with the first surface defining a base of the cavity.

5. The eye mask assembly of claim 4, wherein the cavity tapers inward such that an opening of the cavity is larger in area than the base of the cavity.

6. The eye mask assembly of claim 1, wherein the ring-shaped bolsters are formed with a core and a cover, the core having a substantially rectangular cross-section.

7. The eye mask assembly of claim 6, wherein the cover surrounds the core, and the cover is connected at a connection point to the first surface of the mask, wherein the ring-shaped bolsters flex with respect to the mask at the connection point.

8. The eye mask assembly of claim 6, wherein the core is temporarily deformable and configured to conform to various eye socket and facial shapes.

9. The eye mask assembly of claim 6, wherein the core is a foam material.

10. The eye mask assembly of claim 1, wherein each of the ring-shaped bolsters defines a perimeter sized to be larger than a periphery of a user's eye socket.

11. An eye mask assembly comprising
a mask with a first end and a second end connected by a bridge, and a first surface opposite a second surface;
a strap connecting the first and the second end of the mask; and
a pair of lash-protection rings connected to the mask and extending away from the first surface of the mask, each lash-protection ring having an inner wall and a top end, wherein
the top end of the lash-protection ring forms a skin-contact region of the eye mask assembly;
the inner wall of the lash-protection ring and the first surface of the mask form a cavity; and further wherein
the eye mask assembly is configured to be worn by a user such that
the cavity is configured to be positioned above and around a user's eye socket to prevent a user's eyelashes from contacting the wall or the base of the cavity;
the skin-contact region is configured to contact an area of skin surrounding the user's eye sockets; and
the first surface of the mask is spaced apart from the top end to avoid contact with the user.

12. The eye mask assembly of claim 11, wherein the lash-protection rings are substantially oval or obround in shape.

13. The eye mask assembly of claim 11, wherein an outer wall of each of the lash protection rings extends away from the first surface of the mask at an acute angle with respect to the first surface.

14. The eye mask assembly of claim 9, wherein each of the ring-shaped bolsters defines a cavity with the first surface defining a base of the cavity.

15. The eye mask assembly of claim 14, wherein the cavity tapers inward such that an opening of the cavity is larger in area than the base of the cavity.

16. The eye mask assembly of claim 11, wherein the lash-protection rings are formed with a core and a cover, the core having a substantially rectangular cross-section.

17. The eye mask assembly of claim 16, wherein the cover surrounds the core, and the cover is connected at a seam to the first surface of the mask, wherein the lash-protection rings flex with respect to the mask along the seam.

18. The eye mask assembly of claim 16, wherein the core is temporarily deformable and configured to conform to various eye socket and facial shapes.

19. The eye mask assembly of claim 16, wherein the core is a foam material.

20. The eye mask assembly of claim 11, wherein each of the lash-protection rings defines a perimeter sized to be larger than a periphery of a user's eye socket.
